(19) United States
(12) Patent Application Publication
(10) Pub. No.: US 2015/0050747 A1
(43) Pub. Date: Feb. 19, 2015

(54) DIRECTED DNA CO-POLYMER MASS AMPLIFICATION FOR ssDNA DETECTION

(71) Applicants: Evangelyn C. Alocilja, East Lansing, MI (US); Michael J. Anderson, East Lansing, MI (US)

(72) Inventors: Evangelyn C. Alocilja, East Lansing, MI (US); Michael J. Anderson, East Lansing, MI (US)

(73) Assignee: BOARD OF TRUSTEES OF MICHIGAN STATE UNIVERSITY, East Lansing, MI (US)

(21) Appl. No.: 13/184,998

(22) Filed: Jul. 18, 2011

Related U.S. Application Data
(60) Provisional application No. 61/365,551, filed on Jul. 19, 2010.

(51) Int. Cl. G01N 33/53; G01N 27/447; C07H 21/04

(52) U.S. Cl. CPC G01N 33/5308; C07H 21/04; G01N 27/447
USPC 436/501; 536/23.1; 204/456

(57) ABSTRACT

The disclosure generally relates to the use of a bio-barcode (BBC) method for capture and demonstrates hybridization co-polymerization amplification readout of the BBC as a method of rapid detection of single stranded DNA output with an increased sensitivity for the initial target input to the BBC assay. The BBC assay reporter ssDNA can be modified for rapid readout. Two ssDNA molecules are designed to co-polymerize and then continually hybridize into double stranded DNA. The double stranded DNA can be optically and/or electrically detected. Kits related to the two ssDNA molecules and the BBC assay also are disclosed.

### CROSS-REFERENCE TO RELATED APPLICATION

Priority is claimed to U.S. Provisional Application No. 61/365,551, filed Jul. 19, 2010, the disclosure of which is incorporated herein by reference in its entirety.

### STATEMENT OF GOVERNMENT INTEREST

This invention was made with government support from the Department of Homeland Security under grant number DHS-2007-ST-061-000003. The government has certain rights in the invention.

### REFERENCE TO A SEQUENCE LISTING SUBMITTED IN A COMPUTER READABLE FORMAT

The application contains nucleotide sequences which are identified with SEQ ID NOs. The Sequence Listing provided in computer readable form, incorporated herein by reference in its entirety, is identical to the written copy of the Sequence Listing provided with the application.

### BACKGROUND OF THE DISCLOSURE

1. Field of the Disclosure

The present disclosure is directed to a method for rapidly detecting single-stranded DNA (ssDNA) sequence by co-polymerization mass amplification. The co-polymerization amplification results in a larger double-stranded DNA (dsDNA) molecule, which can be efficiently detected by a DNA binding dye.

2. Brief Description of Related Technology

The single Anthrax attack in 2001 cost the U.S. over $1 billion in decontamination and recuperation efforts. See R. Johnston, “Review of Fall 2001 Anthrax Bioattacks.” vol. 2009, 2007. The five deaths from the 2001 attack may have been preventable if diagnosed and treated immediately after initial exposure. Emergency situations—including bioterrorism events—could be dealt with much more effectively by rapid identification of exposed individuals and result in faster, more targeted treatments with significantly decreased economic costs and loss of life. Current detection strategies do not sufficiently provide for rapid and specific identification for Anthrax. These bacterial detection strategies are broadly grouped into three categories: culture based identification, immunological recognition, and nucleic acid chemistry. Culture techniques provide accurate target identification but require long times (18-96 hours) for identification. See R. T. Noble and S. B. Weisberg, “A review of technologies for rapid detection of bacteria in recreational waters,” J Water Health, vol. 3, pp. 381-92, December 2005. Immunological methods are reliable and accurate but have long production times, limited shelf lives, and unique to manufacturer. Antibodies are not capable of distinguishing between lethal strands of Bacillus anthracis and the non-lethal but closely related Bacillus anthracis Sterne strain. This can lead to false positive detections. Nucleic acid techniques typically rely on PCR and/or hybridization arrays and are very sensitive. However, use is limited by long testing times and system complexity. Biosensor technologies for detection of bacterial microorganisms include conductive polymer nano-wires, see, S. Pal, E. C. Alocilja, and F. P. Downes, “Nanowire labeled direct-charge transfer biosensor for detecting Bacillus species,” Biosens Bioelectron, vol. 22, pp. 2329-36, Apr. 15, 2007, nano-porous silicon, see F. P. Mathew and E. C. Alocilja, “Porous silicon-based biosensor for pathogen detection,” Biosens Bioelectron, vol. 20, pp. 1656-61, Feb. 15, 2005, surface plasmon resonance, see F. C. Dudak, “Rapid and label-free bacteria detection by surface plasmon resonance (SPR) biosensors,” Biotechnology Journal, pp. 1003-1011, 2009, and gold nano-particles, see G. Li, X. Li, J. Wan, and S. Zhang, “Dendrimers-based DNA biosensors for highly sensitive electrochemical detection of DNA hybridization using reporter probe DNA modified with Au nanoparticles,” Biosens Bioelectron, vol. 24, pp. 3281-7, Jul. 15, 2009. Nano-particle based detection of microorganisms has gained attention in recent years because of the need for rapid detection in multiple readout formats for clinical, food safety and defense applications. The 2001 Anthrax attacks are one example where rapid detection would have aided in cleanup and containment efforts and significantly reduced economic costs. A rapid, accurate, inexpensive field deployable biosensor is needed for ensuring domestic safety in crisis situations.

While Bio-barcode assays are very sensitive, but once the amplified single stranded (ssDNA) output is obtained, up to four additional hours of readout time are required, i.e., microarray hybridization, hybridization to gold nanoparticles followed by silver enhancement and reflectance detection. These methods also require large amounts of laboratory equipment that are not readily portable. Recent DNA detection methods are based on modifications to the bio-barcode assay (BCA) which can use either an antibody or DNA-based capture technique and provide amplification. See H. D. Hill and C. A. Mirkin, “The bio-barcode assay for the detection of protein and nucleic acid targets using DTT-induced ligand exchange,” Nat Protoc, vol. 1, pp. 324-36, 2006; B. K. Oh, J. M. Nam, S. W. Lee, and C. A. Mirkin, “A fluorophore-based bio-barcode amplification assay for proteins,” Small, vol. 2, pp. 103-8, January 2006; and S. I. Stoeva, J. S. Lee, C. S. Thaxton, and C. A. Mirkin, “Multiplexed DNA detection with bio-barcoded nanoparticle probes,” Angew Chem Int Ed Engl, vol. 45, pp. 3303-6, May 12, 2006. The system includes two types of particles: magnetic micro-particles (MMP) for capture and separation and gold nano-particles (AuNP) for capture and amplification. For DNA based systems, oligonucleotides are attached to both the MMP and the AuNP. The oligonucleotides on each particle differ and each recognize and capture a different location of the gene of interest. When the target DNA binds, it forms a link between both the MMP and the AuNP. An applied magnetic field attracts the MMPs and the MMP-target-AuNP complexes at the tube wall, allowing for removal of the supernatant, excess AuNPs and sample debris. A second oligonucleotide is attached to the AuNP at 100× ratio to the capture strand and is called the bio-barcode compliment (BCC). Hybridized to the BCC is the reporter DNA strand called the bio-barcode (BBC). The BBC when released, produces 100 copies of short single stranded DNA for each of the original link DNA target molecules in the system and provides the amplification mechanism. After the BBC is released, it is then used to link an AuNP to an array surface through a second round of hybridization. The AuNP is then ‘enhanced’ by silver deposition and finally detected with scanning reflectance measurements. The readout steps for the released BBC require approximately half of the processing time of the entire BCA and limits its for diagnosis in acute situations. J. M. Nam, S. I. Stoeva, and C. A. Mirkin, “Bio-bar-code-based DNA detection with PCR-like sensitivity,” J Am Chem Soc, vol. 126, pp. 5932-3, May 19, 2004.

### SUMMARY

The disclosure relates to the use of the bio-barcode method for capture and demonstrates hybridization co-polymerization amplification readout of the BBC as a method of rapid detection of single stranded DNA output with a sensitivity of 100 μg of initial target input to the BCA. The BCA reporter ssDNA can be modified for rapid readout. Two ssDNA molecules are designed to co-polymerize and then continually hybridize into double stranded DNA. The double stranded DNA can be optically detected using a dye, (e.g., PICOGREEN dye, an intercalating fluorescent dye). Additionally a semi-metallic nano-particle can be conjugated to a dye for electrical reduction/oxidation readout (e.g., SYBR 101 dye). Co-polymerization and dye detection provide rapid readout of ssDNA from BCA with potential for multiplexed detection through electrical detection.

In one aspect, the disclosure relates to a method for making a double-stranded DNA molecule, the method comprising: (a) providing a population of a first single-stranded DNA (ssDNA) oligonucleotide having a first region and a second region in the 5′ to 3′ direction, wherein the first region is 5′ in position relative to the second region; (b) providing a population of a second single-stranded DNA (ssDNA) oligonucleotide having a third region and a fourth region in the 3′ to 5′ direction for hybridization with the first ssDNA oligonucleotide, wherein: (i) the third region is 3′ in position relative to the fourth region; (ii) the second region is complementary to the third region, and the first region is complementary to the fourth region; and (iii) the first region and the fourth region become overhangs when the second region and the third region hybridize together, and the second region and the third region become overhangs when the first region and the fourth region hybridize together; (c) hybridizing the population of the first ssDNA oligonucleotide and the population of the second ssDNA oligonucleotide under a predetermined hybridization condition to form a double-stranded DNA (dsDNA) molecule, the dsDNA molecule having repetitive first ssDNA oligonucleotide units on one strand and repetitive second ssDNA oligonucleotide units on the other strand; and optionally (d) detecting the dsDNA molecule (e.g., optically detecting the dsDNA molecule, electrically detecting the dsDNA molecule, such as in a BBC assay or other method for detecting the dsDNA molecule).

In an embodiment, the part (a) of providing the first ssDNA oligonucleotide comprises: (i) providing a bio-barcode nanoparticle probe comprising: (A) a nanoparticle core, (B) a first binding pair member (e.g., a capture strand; an antibody) immobilized on the nanoparticle core, the first binding pair member being capable of binding to a target analyte (e.g., a target DNA sequence; a target antigen/protein), and (C) the first ssDNA oligonucleotide immobilized on the nanoparticle core; and (ii) releasing the first ssDNA oligonucleotide from the nanoparticle core to provide the population of the first ssDNA oligonucleotide. In a refinement, the target analyte is a target DNA sequence, and the first ssDNA oligonucleotide and the second ssDNA oligonucleotide do not hybridize with the target DNA sequence. In a refinement, the first ssDNA oligonucleotide is directly attached to the nanoparticle core in part (a)(i). In another refinement, the bio-barcode nanoparticle probe further comprises (D) a bio-barcode complement single-stranded DNA (BBC complement ssDNA) oligonucleotide directly attached to the nanoparticle core, the BBC complement ssDNA being capable of hybridization with the first ssDNA; and the first ssDNA oligonucleotide is immobilized on the nanoparticle core in part (a)(i) via conjugation with the BBC complement ssDNA.

In another aspect, the disclosure relates to kit for forming a double-stranded DNA (dsDNA) molecule, the kit comprising: (a) a first single-stranded DNA (ssDNA) oligonucleotide having a first region and a second region in the 5′ to 3′ direction, wherein the first region is 5′ in position relative to the second region; and (b) a second single-stranded DNA (ssDNA) oligonucleotide having a third region and a fourth region in the 3′ to 5′ direction for hybridization with the first ssDNA oligonucleotide, wherein: (i) the third region is 3′ in position relative to the fourth region; (ii) the second region is complementary to the third region, and the first region is complementary to the fourth region; and (iii) the first region and the fourth region become overhangs when the second region and the third region hybridize together, and the second region and the third region become overhangs when the first region and the fourth region hybridize together. In a refinement, the first ssDNA oligonucleotide can be provided in the kit in the form of a bio-barcode nanoparticle probe comprising: (A) a nanoparticle core, (B) a first binding pair member (e.g., a capture strand; an antibody) immobilized on the nanoparticle core, the first binding pair member being capable of binding to a target analyte (e.g., a target DNA sequence; a target antigen/protein), and (C) the first ssDNA oligonucleotide immobilized on the nanoparticle core. The kit can further comprise: (c) a magnetic microparticle probe comprising: (A) a magnetic microparticle core, and (B) a second binding pair member (e.g., a capture strand; an antibody) immobilized on the magnetic microparticle core, the second binding pair member being capable of binding to the target analyte.

Various refinements and embodiments of the disclosed methods and kits are possible. For example, the first ssDNA oligonucleotide and the second ssDNA oligonucleotide each independently can have between 8 to 60 bases (e.g., 16 or 18 to 36, or 22 bases). In a refinement, (i) the first ssDNA oligonucleotide comprises a sequence according to SEQ ID NO: 1 (e.g., a modified BBC target sequence 5′-TTATT CGTAG CGTGA TGCCA AG-3′, where the first region is TTATT CGTAG C and the second region is GTGA TGCCA AG in the 5′-to-3′ direction, respectively, and where the sequence can be further functionalized at either end); and (ii) the second ssDNA oligonucleotide comprises a sequence according to SEQ ID NO: 2. (e.g., a CPH-probe sequence 5′-GCTAC GAATA ACTTG GCATC AC-3′, where the third region is CTTG GCATC AC and the fourth region is GCTAC GAATA A in the 5′-to-3′ direction, respectively). In another refinement, the second region is complementary to the third region by at least 85%, and the first region is complementary to the fourth region by at least 85% (e.g., wherein the second region is less than 100% and at least 85% complementary to the third region, and the first region is less than 100% and at least 85% complementary to the fourth region). In another refinement, the dsDNA molecule has a average length of at least 40 base pairs. In another refinement, the first ssDNA oligonucleotide does not hybridize with itself and the second ssDNA oligonucleotide does not hybridize with itself. In another refinement, a ratio of the first ssDNA oligonucleotide to the second ssDNA oligonucleotide ranges between 0.1 and 10. In an embodiment, the method comprises gradually adding the population of the second ssDNA oligonucleotide to the population of the first ssDNA oligonucleotide. In another embodiment, the method comprises providing the population of the first ssDNA oligonucleotide as a plurality of subsamples to which a different amount of the second ssDNA oligonucleotide is added for each subsample.

An object of the present disclosure is to provide a method for detecting a single-stranded DNA molecule, which comprises the steps of: (a) preparing a population of a first single-stranded DNA (ssDNA) oligonucleotide having a first and a second region in the 5′ to 3′ direction, wherein said first region is 5′ in position relative to said second region; (b) constructing a population of a second single-stranded DNA oligonucleotide having a third and a fourth region in the 3′ to 5′ direction for hybridization with said first ssDNA oligonucleotide, said third region is 3′ in position relative to said fourth region; wherein said second region is complementary to said third region, and said first region is complementary to said fourth region; wherein said first and said fourth regions become overhangs when said second and said third regions hybrid together, and said second and said third regions become overhangs when said first and said fourth regions hybrid together; (c) hybridizing the population of said first ssDNA oligonucleotide and the population of said second ssDNA oligonucleotide under a predetermined hybridization condition to form a double-stranded DNA (dsDNA) molecule, said dsDNA molecule having repetitive first ssDNA oligonucleotide units on one strand and repetitive second ssDNA oligonucleotide units on the other; and (d) detecting said dsDNA using a DNA binding dye.

Another object of the present disclosure is to provide a method of making a double-stranded DNA molecule, which comprises the steps of: (a) preparing a population of a first single-stranded DNA (ssDNA) oligonucleotide having a first and a second region in the 5′ to 3′ direction, wherein said first region is 5′ in position relative to said second region; (b) constructing a population of a second single-stranded DNA oligonucleotide having a third and a fourth region in the 3′ to 5′ direction for hybridization with said first ssDNA oligonucleotide, said third region is 3′ in position relative to said fourth region; wherein said second region is complementary to said third region, and said first region is complementary to said fourth region; wherein said first and said fourth regions become overhangs when said second and said third regions hybrid together, and said second and said third regions become overhangs when said first and said fourth regions hybrid together; and (c) hybridizing the population of said first ssDNA oligonucleotide and the population of said second ssDNA oligonucleotide under a predetermined hybridization condition to form a double-stranded DNA (dsDNA) molecule, said dsDNA molecule having repetitive first ssDNA oligonucleotide units on one strand and repetitive second ssDNA oligonucleotide units on the other.

All patents, patent applications, government publications, government regulations, and literature references cited in this specification are hereby incorporated herein by reference in their entirety. In case of conflict, the present description, including definitions, will control.

Additional features of the disclosure may become apparent to those skilled in the art from a review of the following detailed description, taken in conjunction with the examples, drawings, and appended claims, with the understanding that the disclosure is intended to be illustrative, and is not intended to limit the claims to the specific embodiments described and illustrated herein.

### BRIEF DESCRIPTION OF THE DRAWINGS

For a more complete understanding of the disclosure, reference should be made to the following detailed description and accompanying drawing wherein:

FIG. 1 is a schematic representation of various single-stranded and double-stranded DNA oligonucleotides according to the present disclosure for use in a co-polymerization hybridization and mass amplification process: (a) first single-stranded DNA (ssDNA) oligonucleotide (e.g., usable as a bio-barcode); (b) hybridized conjugate of the first ssDNA and a complementary ssDNA oligonucleotide (e.g., usable as a bio-barcode complement); (c) inversion/rearrangement of the complementary ssDNA oligonucleotide to select a second ssDNA oligonucleotide structure; (d) hybridization coupling between first and second ssDNA oligonucleotides to from a double-stranded DNA (dsDNA) molecule; (e) generalized dsDNA molecule with a plurality of overlapping first and second ssDNA elements with overhang structures for additional hybridization interactions; (f) specific embodiments of a first ssDNA oligonucleotide, a second ssDNA oligonucleotide, a dsDNA molecule, and a complementary ssDNA oligonucleotide; and (g) bio-barcode assay components for use with the disclosed process and components.

FIG. 2 is a gel electrophoresis showing dilution of a 60 bp dsDNA with SYBR 101 and SYBR 101-QD. 1 μg 100 bp ladder (1,2). SYBR 101 stained lanes with 75 ng, 30 ng, and 15 ng of dsDNA (3,5,7) and SYBR 101-QD stained lanes with 75 ng, 30 ng, and 15 ng of dsDNA (4,6,8) respectively. Gel: 2% w/v agarose, 30V, 2 hr in 1×TBE buffer.

FIG. 3 is a gel electrophoresis showing the hybridization banding. 1 μg 100 bp ladder (1) in each lane (1-8) contains 150 ng of ssDNA; target BBC to CPH-probe ratios are 10:1, 5:1, 2:1, 1:1, 1:2, 1:5, and 1:10 respectively for lanes 2-8. Gel: 2% w/v agarose, 30V, 2 hr in 1×TBE buffer.

FIG. 4 is a gel electrophoresis showing dye specificity to the co-polymerized dsDNA products. 1 μg 100 bp ladder (1) stained with SYBR Gold; lanes (2-4) were run with 45 ng of ssDNA; lanes (5-7) were run with 15 ng of dsDNA. SYBR Gold, PICOGREEN and Ethidium.

FIG. 5 is a gel electrophoresis showing the results of hybridized CPH-probe and BBC with PICOGREEN and SYBR 101-QD dyes. 1 μg 100 bp ladder (1), 450 μg of ssDNA at 10:1, 1:1, and 1:10 ratio of probe to target in lanes (2-4) and (5-7), respectively. Lanes (1-4) stained with PICOGREEN and lanes (5-7) stained with SYBR 101-QD. Gel: 2% w/v agarose, 30 V, 2 h in 1×TBE buffer.

FIG. 6 is a graph illustrating the sensitivity of the disclosed detection method as a function of CPH probe amount: (A) ssDNA only; (B-F) increasing amounts of CPH-probe.

FIG. 7 is a graph illustrating the sweeping voltammetry readout of metallic reduction. Peaks are seen at −0.87 and −0.90 V indicating the presence of cadmium in the SYBR101-QD sample.

While the disclosed compositions, kits, apparatus, and methods are susceptible of embodiments in various forms, specific embodiments of the disclosure are illustrated in the drawings (and will hereafter be described) with the understanding that the disclosure is intended to be illustrative, and is not intended to limit the claims to the specific embodiments described and illustrated herein.

### DETAILED DESCRIPTION

The disclosed method and system are directed to the rapid detection of an ssDNA reporter using co-polymerization mass amplification. The system is based on two different ssDNA strands that are designed to partially hybridize, leaving exposed bases at both 5′ and 3′ ends of the double-stranded hybrid for additional hybridization reactions with the ssDNA strands. As known to a person skilled in the art, an ssDNA strand contains a 5′-to-3′ directionality. Two ssDNA strands will bind according to the Watson-Crick base paring principle, i.e., an adenine (A) binds to a thymine (T) and a cytosine binds to a guanine (G). The two strands when bound together have opposite directionality, i.e., they run anti-parallel. This A/T and C/G base paring is highly specific, the mismatching rates of which are very low for relatively short ssDNA sequences. This principle coupled with appropriately designed oligonucleotide probes, as fully described below, can allow for overlap extensions of the designed oligonucleotide in both 3′ and 5′ directions. As a result, a bio-barcode (BBC) ssDNA from a bio-barcode assay (BCA) is allowed to repeatedly co-polymerize/hybridize with a ssDNA probe (CPH probe) to create a larger dsDNA molecule that can be more easily detected than the BBC ssDNA alone. Thus, after the production of the designed BBC ssDNA, the BBC ssDNA can be amplified and detected after recovery from the BCA. For example, the recovered BBC ssDNA can be mixed with pre-measured intercalating DNA dye and the CPH probe to hybridize the BBC ssDNA and CPH probe, for example by heating (e.g., to 95° C.) and then slowly cooling to room temperature, during which co-polymerization hybridization and dye intercalation of dsDNA occurs if the BBC ssDNA is present. The resulting dsDNA can be detected (e.g., in 200 μL microplate well) insofar as intercalation dye that is bound to hybridized dsDNA will fluoresce indicating that BBC ssDNA was present in the sample.

### TERMS

The term “ssDNA” includes a single free strand of polymerized deoxyribonucleic acids consisting of repeated polymer bases of adenine (A), cytosine (C), guanine (G), and/or thymine (T), where each strand has directionality and runs from five prime (5′) to three prime (3′).

The term “dsDNA” includes a complex of two ssDNA strands that are hybridized to each other in a complimentary fashion (adenine:thymine and cytosine:guanine), the two strands run anti-parallel to each other and form a helical structure, such that at any given end a 5′-end from one strand and a 3′-end from another strand are present.

The term “BBC” or “bio-barcode” includes a single strand DNA molecule that is released from a (gold) nanoparticle upon completion of the bio-barcode assay, where the BBC nucleotide base sequence is designed by the user for ease in detection.

The term “BCA” includes bio-barcode assay, a technique for detection of DNA with amplified readout utilizing magnetic micro particles and gold nanoparticles.

The term “oligonucleotide” includes a DNA molecule having from 8 bases to 1000 bases in length and being single stranded.

The term “strand” includes oligonucleotide.

The term “sequence” includes the specific nucleotide base configuration in a linear 5-prime to 3-prime order.

The term “hybridization” includes the pairing of two oligonucleotides together, where non-covalent bonding occurs between adenine and thymine or cytosine and guanine pairs, and the hybridized oligonucleotides are in opposing orientations during hybridization.

The term “probe” includes the oligonucleotide attached to either the magnetic mico-particle or gold nanoparticle designed to hybridize to the DNA of the target organism.

The term “intercalating agent” or “intercalator” includes any small chemical molecule that binds to DNA based on charge and the three dimensional shape of DNA, these agents are often fluorescent or used as carriers for other reporting types of molecules.

The term “co-polymerization” includes the creation of a larger polymer from two different smaller monomer or polymers units in an ordered assembly, where monomers include ssDNA oligonucleotides and polymers include dsDNA molecules formed from ssDNA oligonucleotides.

The term “co-polymerization hybridization” (CPH) includes the process of two DNA sequences attaching together through hybridization in a repeating fashion to create a double stranded DNA strand longer than either of the individual sequences.

The term “CPH-probe” includes the oligonucleotide designed to hybridize to the BBC, where a single hybridization event can include 50% hybridization between any pair of oligonucleotides.

The term “genomic DNA” includes the DNA carried in an organism for normal life giving functions, where the set of DNA is specific and unique to each organism.

The term “Quantum Dot (QD)” includes a small sphere with a cadmium sulfide core and a zinc sulfide shell with a total outer diameter of 5 to 20 nanometer, but other metal compositions and size are possible.

The term “reporter” includes any molecule used to relay information. DNA reporters are oligonucleotides of known sequence, and dye reporters are visible molecules of known output photon wavelength.

The term “readout” includes final detection of information from an assay system.

The term “DNA binding dye” includes a small molecule that non-covalently attaches to a DNA molecule by recognizing a physical property of the DNA.

The term “fluorophore” includes a molecule that absorbs a photon of a shorter wavelength and emits a photon of a longer wavelength.

The term “MMP” includes magnetic micro-particles with diameter of 0.75 to 5 micometer, where the particle can an iron oxide paramagnetic particle that is attracted to an applied magnetic source. The surface of the MMP can be coated/covered with probe DNA oligonucleotides.

The term “AuNP” includes gold nanoparticles, which cab be a solid gold sphere with a diameter of 5 to 50 nanometers. The surface can be covered with both probe DNA (e.g., for target DNA capture) and BBC DNA oligonucleotides.

The term “polymerase chain reaction” (PCR) includes the process of making double stranded copies of either a ssDNA or dsDNA template using an enzymatic assembly process.

The term “complementary” includes a second sequence of DNA bases that mirrors a first sequence, with the second sequence having the following substitutions adenine (A) in place of thymine (T), cytosine (C) in place of guanine (G), thymine (T) in place of adenine (A) and guanine (G) in place of cytosine (C) in an anti-parallel direction relative to the first sequence.

ssDNA and dsDNA Components

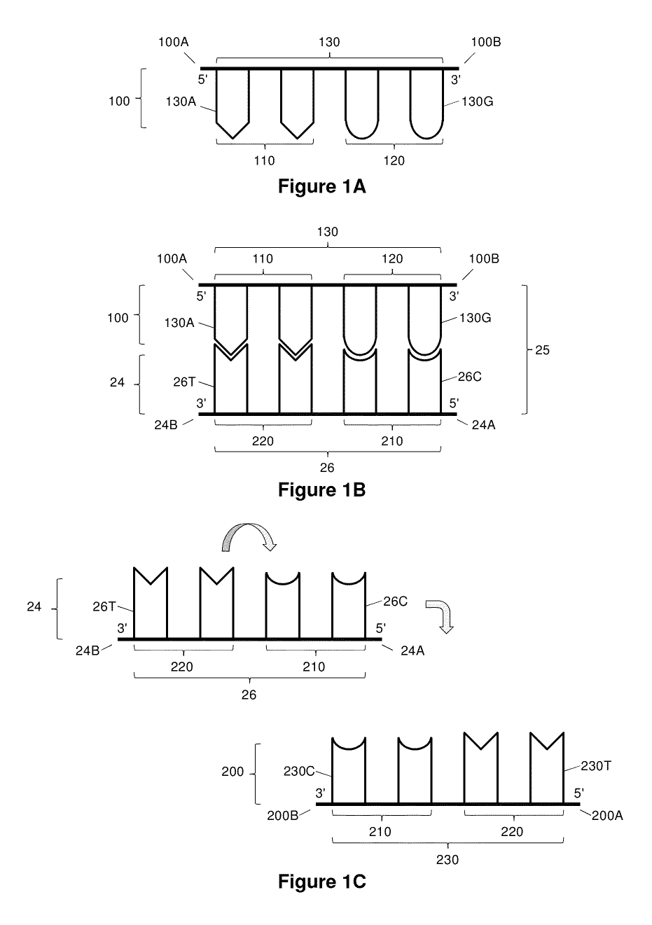

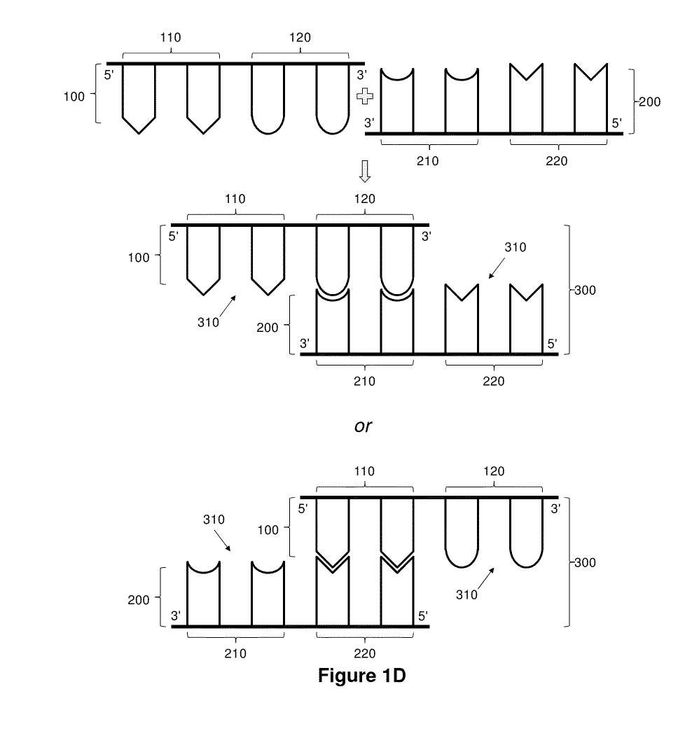

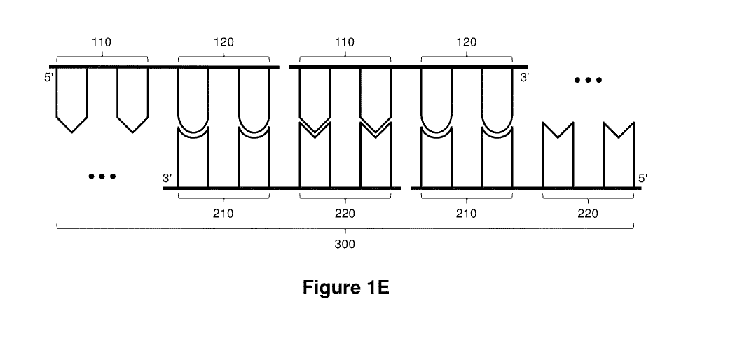

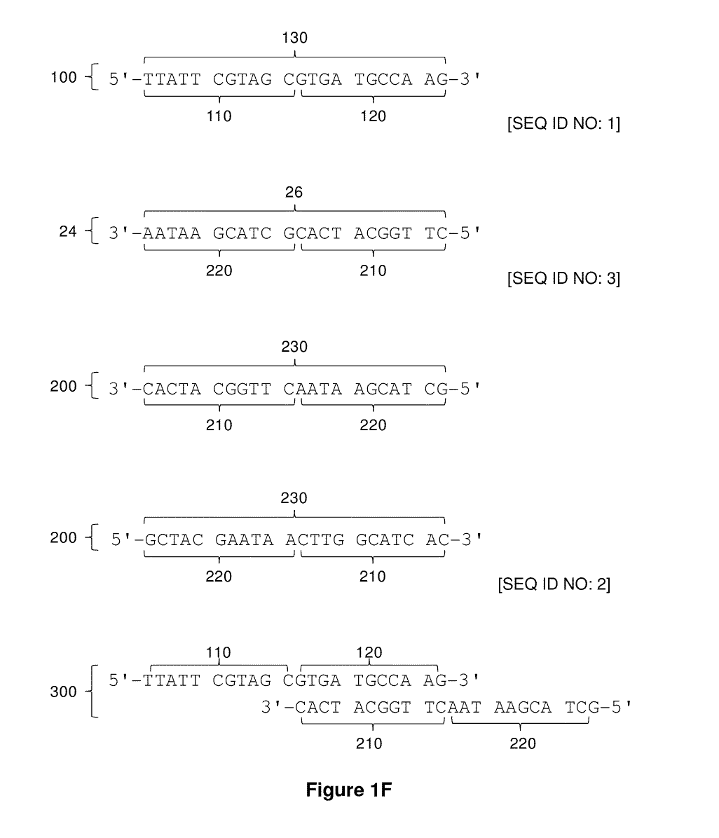

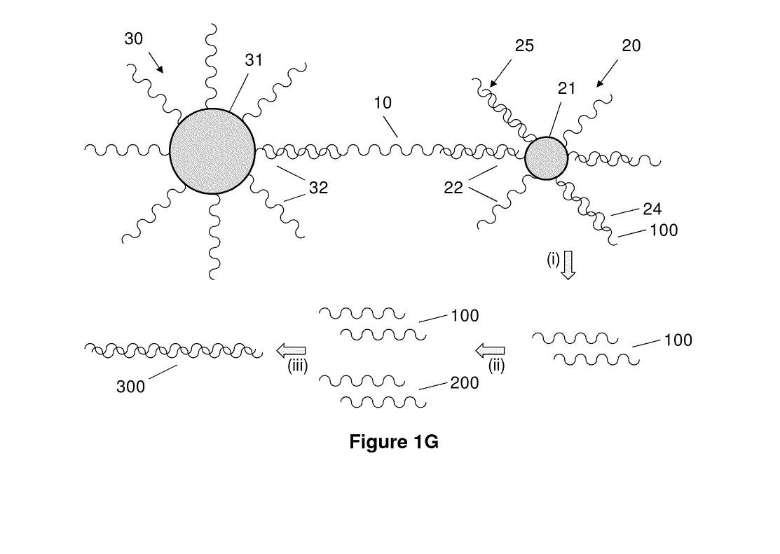

FIGS. 1A-1G illustrate various components including single-stranded and double-stranded DNA oligonucleotides according to the present disclosure for use in a co-polymerization hybridization and mass amplification process (e.g., as a detection/read-out step in a generalized BBC assay).

As illustrated particularly in FIGS. 1A and 1C, the methods, kits, and systems according to the disclosure include a first ssDNA oligonucleotide 100 and a second ssDNA oligonucleotide 200. In the context of a BBC assay, the first ssDNA oligonucleotide 100 can represent the BBC ssDNA, and the second ssDNA oligonucleotide 200 can represent a co-polymerization hybridization (CPH) ssDNA probe according to the disclosure.

The first ssDNA oligonucleotide 100 includes a plurality of bases 130 in an oligonucleotide having a 5′-end 100A and a 3′-end 100B. The plurality of bases 130 is characterized as having two separate regions along its length: a first region 110 and a second region 120, which represent different subsets/subsequences of the first ssDNA oligonucleotide 100. The first region 110 is 5′ in position relative to the second region 120 (e.g., closer to the 5′-end 100A than the 3′-end 100B). As illustrated, the first region 110 and the second region 120 together constitute the entire plurality of bases 130 for the first ssDNA oligonucleotide 100 such that the regions 110, 120 are in sequence without other intervening bases or functional groups. In other embodiments (not shown), however, the first ssDNA oligonucleotide 100 can include other bases in the plurality 130 that are not part of the regions 110, 120 (e.g., between the 5′-end 100A and the first region 110, between the first region 110 and the second region 120, and/or between the second region 120 and the 3′-end 100B). For the purposes of illustration, the plurality of bases 130 is shown as having two adenine bases 130A and two guanine bases 130G such that the illustrated first ssDNA oligonucleotide 100 has the sequence 5′-AAGG-3′, the first region 110 is represented by the subsequence 5′-AA-3′, and the second region 120 is represented by the subsequence 5′-GG-3′.

Analogous to the first ssDNA oligonucleotide 100, the second ssDNA oligonucleotide 200 includes a plurality of bases 230 in an oligonucleotide having a 5′-end 200A and a 3′-end 200B. The plurality of bases 230 is characterized as having two separate regions along its length: a third region 210 and a fourth region 220, which represent different subsets/subsequences of the second ssDNA oligonucleotide 200. The third region 210 is 3′ in position relative to the fourth region 220 (e.g., closer to the 3′-end 200B than the 5′-end 200A). As illustrated, the third region 210 and the fourth region 210 together constitute the entire plurality of bases 230 for the second ssDNA oligonucleotide 200 such that the regions 210, 220 are in sequence without other intervening bases or functional groups. In other embodiments (not shown), however, the second ssDNA oligonucleotide 200 can include other bases in the plurality 230 that are not part of the regions 210, 220 (e.g., between the 3′-end 200B and the third region 210, between the third region 210 and the fourth region 220, and/or between the fourth region 220 and the 5′-end 200A). For the purposes of illustration, the plurality of bases 230 is shown as having two cytosine bases 230C and two thymine bases 230T such that the illustrated second ssDNA oligonucleotide 200 has the sequence 5′-TTCC-3′, the third region 210 is represented by the subsequence 5′-CC-3′, and the fourth region 220 is represented by the subsequence 5′-TT-3′.

The first region 110, second region 120, third region 210, and fourth region 220 are relative terms in that they relate the manner in which the first ssDNA oligonucleotide 100 and the second ssDNA oligonucleotide 200 interact (e.g., under hybridization conditions). The specific base sequences that are selected for the oligonucleotides 100, 200 and their respective regions 110, 120, 210, 220 are not particularly limited; arbitrary sequence selections are possible to provide the desired interaction between the oligonucleotides 100, 200. In particular, the sequences are selected such that the second region 120 is complementary to the third region 210, and the first region 110 is complementary to the fourth region 220. Thus, each oligonucleotide 100, 200 is capable of hybridizing with the opposing oligonucleotide (but not with itself) to form a conjugate double-stranded DNA (dsDNA) molecule 300. As illustrated in FIG. 1D, the first region 110 and the fourth region 220 become overhangs 310 (i.e., protruding, non-conjugated bases in an otherwise double-stranded DNA molecule that are capable of further binding/hybridization interactions) when the second region 120 and the third region 210 hybridize together. As alternatively illustrated in FIG. 1D, the second region 120 and the third region 210 become overhangs 210 when the first region 110 and the fourth region 220 hybridize together. As seen in FIG. 1E, additional copies of the oligonucleotides 100, 200 can continue to add to the already hybridized dsDNA 300 structure, thus producing dsDNA fragments that are much longer than those produced by 1:1 hybridization (e.g., as illustrated in FIG. 1B, described below).

Complete complementarity in the hybridization regions 110, 120, 210, and 220 where the first and second ssDNA oligonucleotides 100, 200 hybridize is not required in the disclosed methods and systems under a given condition. One or more bases of the oligonucleotides 100, 200 in the hybridization regions 110, 120, 210, and 220 may be altered or linked to another chemical compound (e.g., a dye for optical detection or a quantum dot for electrical detection) to provide additional functionality to the oligonucleotides 100, 200 beyond their ability to hybridize with each other. Suitably, the oligonucleotides 100, 200 may be complementary to each other in a range between 85% and 100%, inclusively (e.g., the second region 120 and third region 210 are 85%-100% complementary to each other and/or the first region 110 and fourth region 220 are 85%-100% complementary to each other, where the degrees of complementarity need not be the same for the two pairs).

The first ssDNA oligonucleotide 100 and the second ssDNA oligonucleotide 200 having selected sequences can be produced using methods generally known to those skilled in the art. Suitably, the oligonucleotide 100, 200 sequences may be selected to have from 8 bases to 60 bases or from 16 or 18 bases to 36 bases (e.g., 22 bases as in the specific example below). Suitable design criteria for the oligonucleotide 100, 200 sequences include: (i) 40-60% guanine (G) or cytosine (C) content; (ii) 18-36 bases in total length for each ssDNA oligonucleotide 100, 200; (iii) equal lengths between the two oligonucleotides 100, 200; (iv) 50% bonding between two individual oligonucleotide 100, 200 molecules (e.g., the regions 110, 120, 210, 220 have equal lengths so that each region represents half of the total length of its respective ssDNA molecule); (v) a 50% hybridization ratio (Tm) in the range 21-50° C.; (vi) a “G” or “C” with 2 location from either end of both oligonucleotide 100, 200 molecules; and/or (vii) no secondary structure formation occurs for the oligonucleotides 100, 200 either individually or when mixed. Thus, when the first ssDNA oligonucleotide 100 and the second ssDNA oligonucleotide 200 hybridize, 50% of each oligonucleotide is hybridized to the other molecule creating ssDNA-dsDNA-ssDNA regions in the resulting dsDNA molecule 300. In other embodiments, however, the oligonucleotides 100, 200 need not be divided into equal-length regions; for example, each region 110, 120, 210, 220 can independently have a length that is at least 20%, 30%, 40%, or 50% and/or up to 50%, 60%, 70%, or 80% of the length of its respective ssDNA molecule (although the complementary regions 110, 220 and 120, 210 are suitably selected to have the same or similar lengths). Suitably, the oligonucleotide 100, 200 sequences are selected so that hybridization only occurs between the complementary regions 110, 220 and 120, 210 (e.g., the first ssDNA oligonucleotide 100 does not hybridize with itself, the second ssDNA oligonucleotide 200 does not hybridize with itself, and/or the oligonucleotides 100, 200 do not hybridize with a target DNA sequence for a given assay (or more generally with any known genomic DNA sequence), where the sequences are selected to limit or prevent such binding with any substantial degree of complementarity).

Oligonucleotides 100, 200 having the desired sequences generally can be custom ordered from commercial sources (e.g., available from Integrated DNA Technologies; Coralville, Iowa), and such oligonucleotides can be selected to include further functional modifications (e.g., 5′-end and/or 3′-end modification to facilitate ssDNA attachment or ssDNA/dsDNA detection, such as with a thiol functional group or fluorescent dye).

FIGS. 1B and 1C illustrate a methodology by which a structure for the second ssDNA oligonucleotide 200 can be selected based on a given/selected structure for the first ssDNA oligonucleotide 100. FIG. 1B illustrates a third ssDNA oligonucleotide 24 that is complementary to the first ssDNA oligonucleotide 100 and is shown in a hybridized conjugate 25 between the two oligonucleotides 24, 100 (e.g., for use in a BBC assay where first ssDNA oligonucleotide 100 represents the BBC ssDNA and the third ssDNA oligonucleotide 24 represents the BBC complement ssDNA, such as might be used to attach the BBC ssDNA to a gold or other nanoparticle substrate). The third ssDNA oligonucleotide 24 includes a plurality of bases 26 in an oligonucleotide having a 5′-end 24A and a 3′-end 24B. For the purposes of illustration, the plurality of bases 26 is shown as having two cytosine bases 26C and two thymine bases 26T such that the illustrated third ssDNA oligonucleotide 200 has the sequence 5′-CCTT-3′. As shown, the third ssDNA oligonucleotide 24 includes a third region 210 and a fourth region 220 that are the same as those in the second ssDNA oligonucleotide 200, albeit in different positions (i.e., the third region 210 is 5′ in position relative to the fourth region 220). FIG. 1C illustrates this inversion of the regions 210, 220 in the third ssDNA oligonucleotide 24 to yield the second ssDNA oligonucleotide 200 (i.e., the regions 210, 220 have the same 5′-3′ subsequence ordering in both oligonucleotides 24, 200, but the relative positions of the regions 210, 220 in the two oligonucleotides 24, 200 are switched). This methodology is also illustrated in FIG. 1F for the specific BBC ssDNA oligonucleotide 100 (SEQ ID NO: 1), the CPH probe ssDNA oligonucleotide 200 (SEQ ID NO: 2), the BBC complement ssDNA oligonucleotide 24 (SEQ ID NO: 3), and the resulting dsDNA molecule 300 described in more detail in the examples below.

FIG. 1G illustrates the first and second ssDNA oligonucleotides 100, 200 in the context of a BBC analysis system. The system includes a bio-barcode nanoparticle probe 20 that includes a nanoparticle core 21, a first capture strand 22 immobilized on the nanoparticle core 21, and the first ssDNA oligonucleotide 100 immobilized on the nanoparticle core 20 (e.g., where the first ssDNA oligonucleotide 100 serves as a bio-barcode reporter in the BBC analysis system). The nanoparticle core 21 is suitably a gold nanoparticle (AuNP), but can more generally be any nano-sized particulate substrate (e.g., diameter or other characteristic size on the order of 5 nm to 50 nm) to which the probe components can be immobilized. The first capture strand 22 is generally an ssDNA oligonucleotide and is capable of binding to a target DNA sequence 10 (e.g., the first capture strand 22 is complementary to and capable of hybridizing with a portion of a target ssDNA sequence 10, such as a 5′-end of the target DNA 10 for a 3′-first capture strand 22 probe or vice versa). In various embodiments, the target DNA 10 is genomic DNA of a target pathogen such as a virus or a bacteria. The first capture strand 22 can be immobilized on the nanoparticle core 21 by any convenient means known to the skilled artisan (e.g., via a thiolated 3′- or 5′-terminal functional group for attachment to various substrates such as AuNPs). Similar to the first capture strand 22, the first ssDNA oligonucleotide 100 can be directly immobilized on the nanoparticle core 21 by any convenient means known to the skilled artisan (e.g., via a thiolated 3′- or 5′-terminal functional group for attachment to various substrates such as AuNPs). In the embodiment illustrated in FIG. 1G, the first ssDNA oligonucleotide 100 is indirectly immobilized on the nanoparticle core 21 via hybridization to a BBC complement ssDNA 24 (e.g., shown as a conjugate 25 between the BBC complement ssDNA 24 and the first ssDNA oligonucleotide 100), where the complement 24 is itself directly attached to the nanoparticle core 21 (e.g., also via a thiolated 3′- or 5′-terminal functional group).

The BBC analysis system can further include magnetic microparticle probe 30 that includes a magnetic microparticle core 31 and a second capture strand 32 immobilized on the magnetic microparticle core 31. The magnetic microparticle core 31 is suitably an iron oxide paramagnetic particle (e.g., diameter or other characteristic size on the order of 0.75 μm to 5 μm) that is attracted to an applied magnetic source. The surface of the magnetic microparticle core 31 can be functionalized to promote attachment of the second capture strand 32 to the core 31 (e.g., amine functionalization). Similar to the first capture strand 22, the second capture strand 32 is generally an ssDNA oligonucleotide and is also capable of binding to a target DNA sequence 10 (e.g., the second capture strand 32 is complementary to and capable of hybridizing with a different portion of the target ssDNA sequence 10 relative to the first capture strand 22, such as a 3′-end of the target DNA 10 for a 5′-second capture strand 32 probe or vice versa). The second capture strand 32 can be immobilized on the microparticle core 31 by any convenient means known to the skilled artisan (e.g., via a thiolated 3′- or 5′-terminal functional group for attachment to various substrates such as amine-functionalized microparticles using a suitable cross linker such as sulfo-SMCC).

In other embodiments, the first capture strand 22 and the second capture strand 32 can more generally be a first binding pair member 22 and a second binding pair member 32 that are capable of specific binding to a target analyte (e.g., distinct portions thereof) other than a target DNA sequence. For example, the binding pair members 22, 32 can be antibodies (immunoglobulins) that are complementary to the same antigen analyte (e.g., different portions thereof), such as a protein, an enzyme, or a bacteria/virus (e.g., being capable of specific binding to different surface proteins thereof).

FIG. 1G illustrates a nanoparticle probe 20-target DNA 10-magnetic microparticle probe 30 conjugate commonly formed in a BBC analysis. The conjugate is generally formed by mixing or otherwise combining the nanoparticle probe 20 and the magnetic microparticle probe 30 with a sample containing the target DNA 10 under suitable hybridization conditions, as is generally known to the skilled artisan, in particular in the context of a BBC analysis. A sample generally includes an aliquot of any matter containing, or suspected of containing, the target analyte (e.g., as the DNA sequence 10 or more generally any analyte such as the target antigen or protein). For example, samples can include biological samples, such as samples from taken from animals (e.g., saliva, whole blood, serum, plasma, urine, tears, and the like), cell cultures, plants; environmental samples (e.g., water); and industrial samples. Samples may be required to be prepared prior to analysis according to various known methods. For example, samples may require extraction, dilution, filtration, centrifugation, and/or stabilization prior to analysis. For the purposes herein, “sample” can refer to either a raw sample as originally collected or a sample resulting from one or more preparation techniques applied to the raw sample.

The disclosure also relates to various kits including the first and second ssDNA oligonucleotides 100, 200. The oligonucleotides 100, 200 are generally provided separately (e.g., in separate sealed containers) in any convenient form for storage and/or eventual use (e.g., in a liquid (aqueous) suspension or solution, or in a dried or lyophilized form). In some embodiments, the first ssDNA oligonucleotide 100 can be provided in the form of the bio-barcode nanoparticle probe 20 with the oligonucleotide 100 immobilized thereon, as described above (e.g., also in a liquid suspension or solution, or in a dried or lyophilized form). In this case, the bio-barcode nanoparticle probe 20 can be provided in a form that is already specific a particular target analyte based on the particular first capture strand 22 immobilized thereon. In another embodiment, the kit can additionally include the magnetic microparticle probe 30 specific to the same target analyte in a third separate container (e.g., also in a liquid suspension or solution, or in a dried or lyophilized form).

Formation and Detection of dsDNA Components

In a general method for forming the dsDNA molecule 300, separate populations of the first and second ssDNA oligonucleotides 100, 200 (e.g., liquid (aqueous) suspensions or solutions, or dried or lyophilized forms of the oligonucleotides 100, 200) are combined/mixed under conditions sufficient to hybridize the oligonucleotides 100, 200. While the resulting dsDNA molecule 300 can be a dimer molecule as illustrated in FIG. 1D, the hybridization product suitably includes dsDNA molecules 300 having a plurality of repetitive first ssDNA oligonucleotide 100 units on one strand and a plurality of repetitive second ssDNA oligonucleotide 200 units on the other strand as illustrated in FIG. 1E. The hybridization product typically contains a plurality of dsDNA molecules 300 with a distribution of lengths/molecular weights along with (potentially) remaining/excess non-conjugated first and second ssDNA oligonucleotides 100, 200. The average/median length of the dsDNA molecule 300 can be about 40-60 base pairs (e.g., about 2-3 ssDNA units), such as when one of the oligonucleotides 100, 200 is present in excess relative to the other (e.g., a 10:1 excess of either), and the average/median length of the dsDNA molecule 300 can be greater than about 40-60 base pairs as the oligonucleotides 100, 200 are present in a relative ratio that is closer to 1 (e.g., having an average length of about 100-200 (for example 160) base pairs and/or having a maximum length of about 200-300 (for example 240) base pairs in an embodiment, such as when the ratio is about 1). For example, the length of the dsDNA molecule 300 can be at least 2, 3, 5, 10, 20, or 50 ssDNA units (or at least 40, 60, 100, 200, 400, or 1000 base pairs) and/or up to 5, 8,10, 15, 20, 30, 50, or 100 ssDNA units (or up to 100, 160, 200, 300, 400, 600, 1000, or 2000 base pairs), where the length (e.g., as a double-strand equivalent) can be an average size, a median size, or a range containing all or substantially all (e.g., 5%-95% cumulative percentiles, 10%-90% cumulative percentiles) of the dsDNA molecules 300 in the distribution (e.g., number- or weight-based).

The first and second ssDNA oligonucleotides 100, 200 are suitably combined during hybridization in relative amounts that promote the formation of longer dsDNA molecules 300 (i.e., a higher average number of ssDNA units per chain). For example, the ratio (e.g., molar or weight ratio) of the first ssDNA oligonucleotide 100 to the second ssDNA oligonucleotide 200 can range between 0.1 and 10 (e.g., at least 0.1, 0.2, 0.5, or 1 and/or up to 1, 2, 5, or 10). The ratio can be applied to the ssDNA oligonucleotides 100, 200 in populations prior to their combination, just after their combination (e.g., prior to any substantial hybridization), and/or after hybridization (e.g., including the units in the dsDNA molecules and free, unhybridized ssDNA units). In an embodiment, the second ssDNA oligonucleotide 200 (e.g., as a CPH probe) can be gradually or step-wise added to the first ssDNA oligonucleotide 100 (e.g., as a BBC, both before and after at least some hybridization has occurred) to increase the length of the resulting dsDNA molecule 300. For example, the second ssDNA oligonucleotide 200 is added in an amount such as up to 1, 2, 5, 10 times a theoretical maximum of the first ssDNA oligonucleotide 100 potentially liberated from nanoparticles used in a BBC analysis to form the population of the first ssDNA oligonucleotide 100. Alternatively or additionally, the population of the first ssDNA oligonucleotide 100 can be subdivided into a plurality of subsamples to which varying amounts of the second ssDNA oligonucleotide 200 can be added (e.g., at levels of at least 0.01, 0.02, 0.05, 0.1, 0.2, or 0.5 and/or up to 0.1, 0.2, 0.5, or 1 times the theoretical maximum of the first ssDNA oligonucleotide 100). Outside the context of a BBC analysis, such gradual or variable addition of one ssDNA oligonucleotide having a known amount or concentration can be applied when the other ssDNA oligonucleotide has an unknown amount or concentration.

The hybridization conditions for forming the dsDNA conjugates are not particularly limited and are generally known to the skilled artisan. Suitably, an aqueous mixture or suspension containing the first and second ssDNA oligonucleotides 100, 200 (e.g., in a physiological buffer, such as Tris-Saline EDTA at pH 7.4) is heated to a temperature sufficient to initiate hybridization (e.g., at least 25° C., 50° C., or 75° C. and/or up to 95° C.; holding at the maximum heating temperature for 1 min to 5 min) and then allowed to cool (e.g., to room temperature, such as 20° C. to 25° C.), during which time further hybridization occurs. As described below, additional reagents such as dyes for the detection of the formed dsDNA can be added to the mixture of oligonucleotides 100, 200 during hybridization.

In the context of a BBC assay, the first ssDNA oligonucleotide 100 can be provided to its hybridization medium by releasing the first ssDNA oligonucleotide 100 from a bio-barcode nanoparticle probe 20 to which it is immobilized. The nanoparticle probe 20 can be free in a liquid mixture, or it can be part of a nanoparticle probe 20-target DNA 10-magnetic microparticle probe 30 conjugate as illustrated in FIG. 1G. The first ssDNA oligonucleotide 100 can be released from the nanoparticle probe 20 by methods generally known in the art, for example depending on the manner of immobilization of the oligonucleotide 100 on the nanoparticle core 21. For example, when the first ssDNA oligonucleotide 100 is immobilized on the nanoparticle core 21 via conjugation with the BBC complement ssDNA 24, the oligonucleotide 100 can be released by de-hybridization, such as by heating the probe 20 with the conjugate 25 in (high purity) deionized water. When the first ssDNA oligonucleotide 100 is directly attached to the nanoparticle core 21 by a thiol functional group, the oligonucleotide 100 can be released with a ligand exchange method (e.g., using 1,4-dithio-DL-threitol (DTT) as in Hill (2006)). The released oligonucleotide 100 population is suitably separated from the nanoparticle probe 20 (or a conjugate thereof) is prior to its combination with the oligonucleotide 200 population. Thus, as generally shown in FIG. 1G, the first ssDNA oligonucleotides 100 are (i) released from nanoparticle probe 20, (ii) combined with the population of the second ssDNA oligonucleotides 200, and then (iii) hybridized to form the dsDNA molecules 300.

In an extension of the general method for forming the dsDNA molecules 300, the dsDNA molecules 300 further can be detected (e.g., where a positive detection of the dsDNA molecules 300 correlates to the presence of a target analyte in an original unknown sample, for example in a BBC analysis). As described in detail below, the method of detection is not particularly limited, but it can include optically detecting the dsDNA molecule 300 (e.g., using a DNA binding dye such as an intercalating fluorophore) or electrically detecting the dsDNA molecule 300 (e.g., using a metallic or semi-metallic quantum dot such as a quantum dot attached to an intercalating agent).

The larger dsDNA molecules 300 created according to an embodiment of the present disclosure can be detected more efficiently than those generated through the standard 1:1 hybridization of ssDNA when using DNA binding dyes. Standard direct fluorescence readout from the BCA normally involves the attachment of a single fluorophore (commonly a fluorescein-like molecule) to each BBC. The recovered fluorescein labeled BBC is then directly read in a microwell well. In contrast, an intercalating agent, which produces a detectable signal such as fluorescence when bound to dsDNA, is used in the present disclosure to indicate the presence of the targeted sequence that is hybridized through the present co-polymerization hybridization (CPH) methodology. To demonstrate the detection efficiency of the present disclosure, the co-polymerization hybridization results of the present disclosure are compared to the standard direct readout of BCA, where CPH readout shows higher signal intensity and better signal to noise than standard direct BBC readout.

Electrical detection provides a second platform for readout, automation potential and multiplexing capabilities. Electrical readout can be conducted in full daylight without diminished sensitivity and can be multiplexed more simply by using different metals in Quantum Dots (QD) than choosing non-spectrally overlapping fluorphores. According to one embodiment of the present disclosure, semi-metallic QDs may be attached to an intercalating agent or an intercalator. A QD will be associated with the dsDNA when an intercalator binds to the dsDNA after hybridization, because the QD is chemically attached to the dye that is hydrogen bonded to the DNA. Detection of the dsDNA can be carried out by association, i.e., detecting the QD via electrical detection, instead of by fluorescence via the intercalator. The QDs allow for multiplexed readout and electrical detection via electrical reduction/oxidation with sweeping voltammetry by using different metallic compositions. Each metal species in a QD has a unique voltage peak during sweeping voltamatry. In one example according to the present disclosure, the limit of detection with a 3.3× signal to noise ratio was about 100 pg of initial target using about 25 ng of ssDNA CPH-probe for detection of the released BBC from the bio-barcode assay.

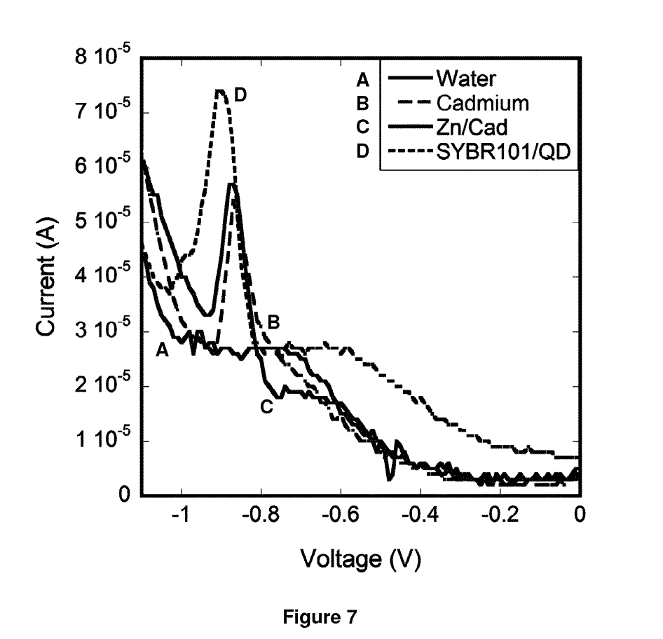

By way of example, liquid samples (20 μL) were dissolved in 80 μL, of 1.0 M hydrochloric acid for 10 minutes and applied to a screen printed carbon electrode (SPCE). The SPCE consists of a carbon counter electrode and a silver/silver chloride reference electrode for measurement. Samples undergo stripping at +1.5 V for 120 seconds before measurement. Sweeping voltammetry was run from +1.5 V to −1.5 V. FIG. 7 plots the current versus voltage for the samples. Cadmium chloride (1 mM; line b) and zinc chloride (1 mM; line c) were used as controls. A peak was not seen for zinc, but the zinc/cadmium mixture had a reduction peak at −0.87 V, the same peak as the cadmium only sample. When the SYBR 101-QD sample was tested, a reduction peak was seen at −0.90 V, indicating the presence of cadmium from the QDs. The ability to detect the cadmium from the SYBR-QD dye presents the ability for future electrochemical readout methods.

In one embodiment of the present disclosure, the effect of CPH-probe to BBC ratio on hybridization was investigated to determine optimal polymerization reaction conditions. Gel electrophoresis, shown in FIG. 3, was used to confirm hybridization had occurred. The BBC to CPH-probe ratios (10:1 5:1 2:1 1:1 1:2 1:5 1:10) were tested to determine the ratio that resulted in formation of the longest dsDNA fragments and stained with SYBR Gold. SYBR Gold was used since it allows the visualization of both dsDNA products and the free ssDNA BBC and CPH-probes. The small bands under lanes 2, 3, and 4 are the excess BBC and the similarly shaped bands in lanes 6, 7, and 8 are excess CPH-probe. Generally, an excess of CPH-probe resulted in hybridized products with higher concentrations of longer fragments, (lanes 6-8 v. 2-4). This increase in signal from the conditions with excess CPH-probe is thought to occur because the CPH-probe's sequences vary within the two distinct regions and result in different hybridization strengths. An increase in binding strength resulted in more efficient and sensitive detection of BBC when the CPH-probe is in excess. With a 10× excess of either the BBC or CPH-probe, the majority of product was 40-60 bp in length. A 1:1 ratio of BBC to CPH-probe yielded the longest double stranded DNA fragments and was selected as the theoretical optimal hybridization condition.

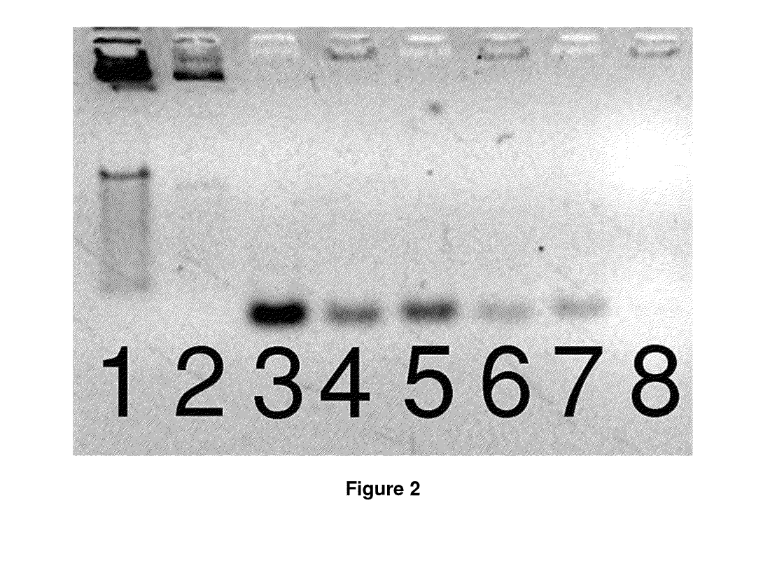

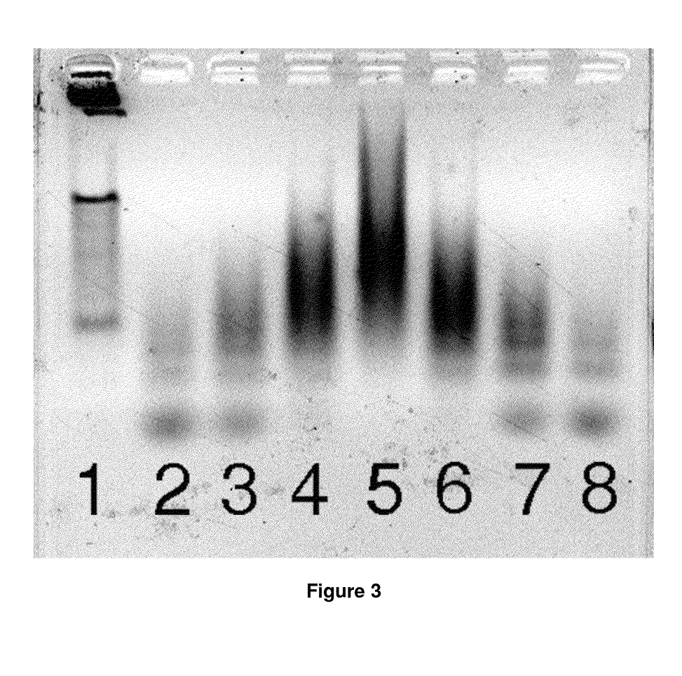

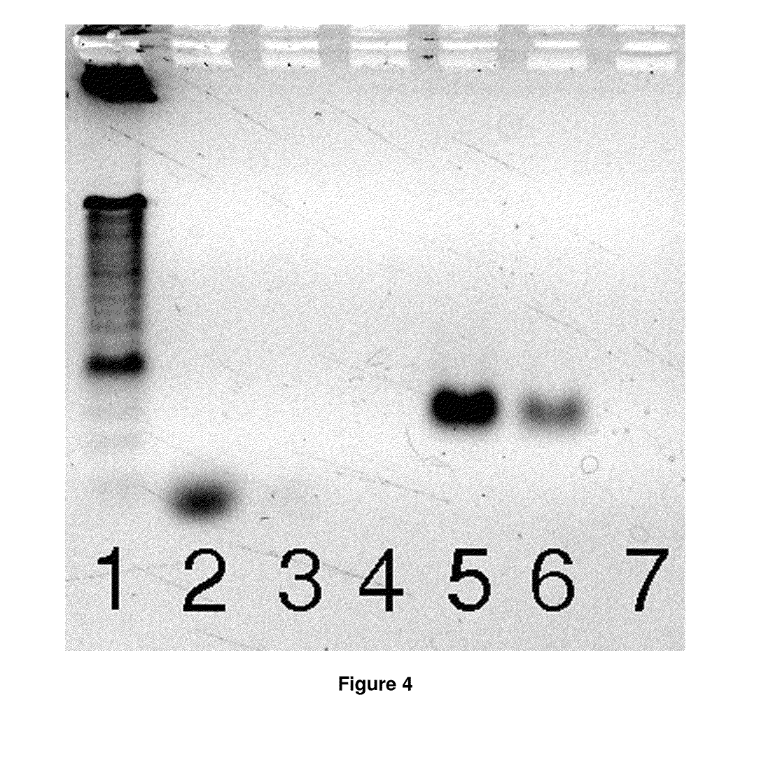

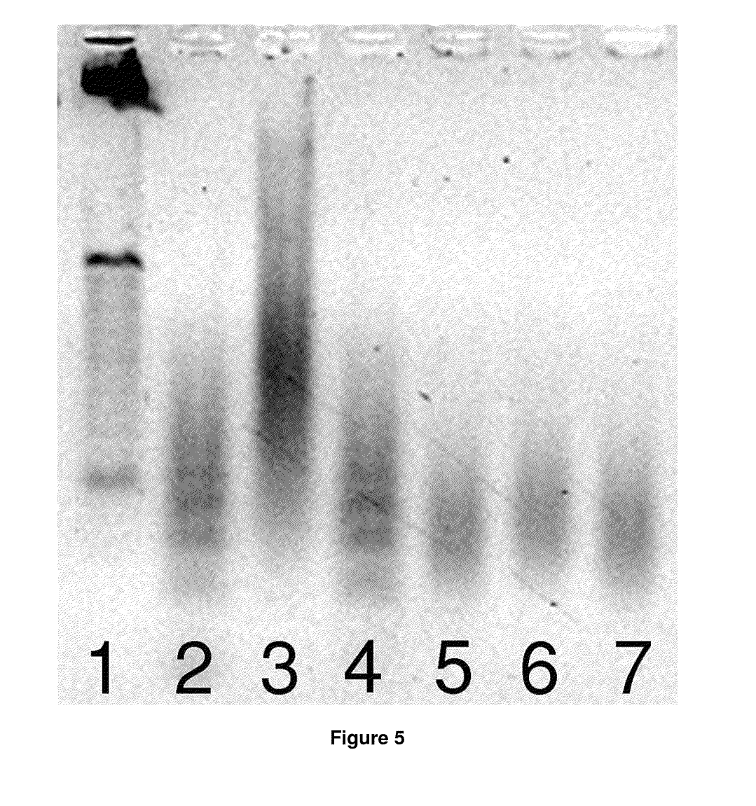

In another embodiment of the present disclosure, gel electrophoresis was used to verify that only the co-polymerized dsDNA product fluoresced using the two experimental dyes—SYBR 101 and PICOGREEN (λₑₓ=480 nm, λₑₘ=520 nm). SYBR Gold (λₑₓ=300 nm, λₑₘ=537 nm) provided a positive control—fluorescing when bound to either dsDNA or ssDNA. Ethidium bromide (λₑₓ=302 nm, λₑₘ=595 nm) was used as a standard for dsDNA fluorescence intensity comparison. Upon hybridization with 2:1 CPH-probe to BBC ratio, fluorescence was observed for the PICOGREEN and SYBR 101 dyes as seen in FIG. 4. The stained lanes (4, 7) were below the detection limit of ethidium bromide in this system. The presence of ssDNA CPH-probe or ssDNA BBC were detected using SYBR Gold (lane 2) but not with SYBR 101 or PICOGREEN (lanes 3, 4). SYBR Gold stained co-polymerized dsDNA and excess ssDNA with the greatest signal intensity (lanes 2, 5). All of the dyes fluoresced when bound to dsDNA. The intensity of PICOGREEN fluorescence (lane 6) was greater than the SYBR 101-QD system (lane 7). The lower intensity of the SYBR 101-QD system was thought to be a result of QD absorption of the excitation signal and possible quenching effects as seen in previously in the SYBR 101 v. SYBR 101-QD comparison. FIG. 5 shows the results of hybridized CPH-probe and BBC with PICOGREEN and SYBR 101-QD dyes. Signal intensities were quantified with QUANTITY ONE imaging software (BioRad; Hercules, Calif.) and PICOGREEN exhibits twice the fluorescence of SYRB 101-QD when either the CPH-probe or the BBC is in ten times excess (lanes 2, 4 v. lanes 5, 7) and a four times increase in intensity for the 1:1 ideal ratio (lanes 3, 6). The lane stained with the SYBR 101-QD (5-7) have migrated farther on the gel than the lanes stained with PICOGREEN (lanes 2-4) in FIG. 5. This weaker signal and faster migration may be the result of the SYBR 101-QD dye having less affinity for DNA binding than PICOGREEN and the greater charge as a result of the attached QD.

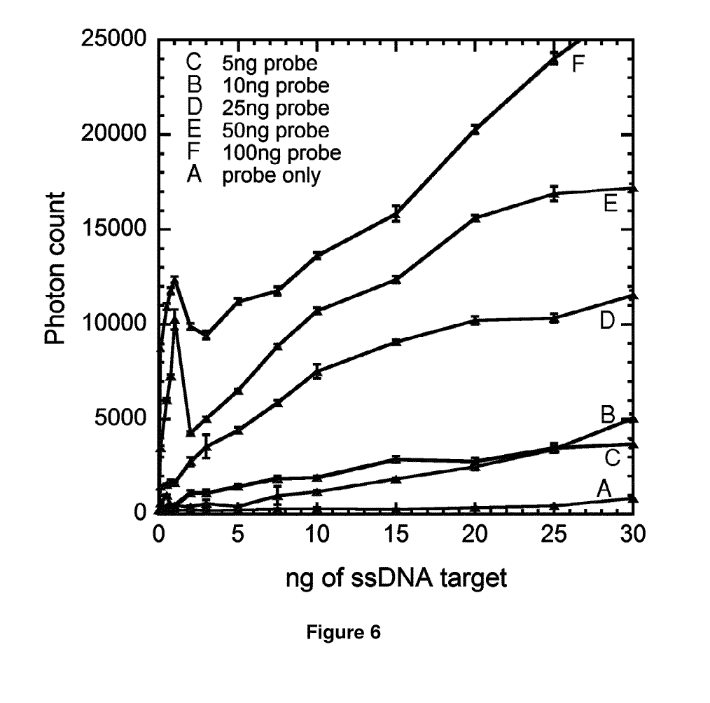

To determine CPH-probe sensitivity for detection of the BBC after the BCA, the CPH-probe to BBC ratio was varied. During co-hybridization amplification conditions, excess CPH-probe or BBC conditions are more probable when a sample of unknown concentration was used for the BCA. Limits of detection for the BBC were carried out with the CPH-probe in excess. Hybridization was measured by photon counting the fluorescence of PICOGREEN bound dsDNA as shown in FIG. 6. Negligible signal was observed for samples that contained only ssDNA CPH-probe which was seen in FIG. 6 (line a). Increased amounts of CPH-probe results show a stronger signal with the photon count increasing for most values of ssDNA target in FIG. 6. With a fixed amount of CPH-probe, an increase in the BBC (ssDNA target) also resulted in increased signal in majority of cases (lines b-f in FIG. 6).

At high CPH-probe concentrations (FIG. 6, 100 ng and 50 ng) a pronounced local maxima was seen between BBC amounts of 0.1-2 ng. These local maxima may be the result of the PICOGREEN during the initial hybridization. PICOGREEN is a minor groove intercalator. During the cooling phase from 95° C., initial hybridization occurs with PICOGREEN binding to and stabilizing the duplexes. PICOGREEN will bind to the dsDNA region at random, and any PICOGREEN binding near the end of a hybridized region has a higher chance to disassociate. At low BBC amounts, there is sufficient CPH-Probe available to bind to the newly exposed overhang once the dye molecule disassociates. This effect may be overcome at higher BBC level as a result of more initial hybridization events occurring. With increased BBC amounts, the chance of larger dsDNA fragments increases as does the signal.

Determination of optimal CPH-probe conditions was based on the region of CPH-probe that is beyond the local maximum observed. The CPH-probe amount of 25 ng exhibited the best direct signal to target relationship with a signal to noise ratio (SNR) of 3:1. The limit of BBC detection for 25 ng of CPH-probe was 100 pg in a 100 μL sample (1× PICOGREEN, 1× Tris-Saline EDTA, pH 7.4). A working limit of detection at 1 ng of BBC is used for the full BCA detection.

Standard BCA fluorescence readout of 6-FAM labeled BBC yields a detection limit of 1 ng of target DNA at 2.3× signal to noise ratio when the readout of the assay is done using only the 6-FAM labeled BBC. The signal intensity before co-polymerization amplification of the 6-FAM labeled BBC was less than 1000 detections per 0.1 seconds and relatively weak. When the entire assay was run, the CPH enhanced BCA can detect 100 pg of target DNA, a 10× increase in detection. The co-polymerization allows for a stronger (1460 detections per 0.1 s) and more sensitive signal for detection of the modified BBC sequence while only adding 20 minutes of readout time.

### EXAMPLES

The following examples illustrate various compositions, apparatus, and methods according to the disclosure for forming and detecting dsDNA molecules (e.g., in a BCA analysis), but are not intended to limit the scope of the claims appended hereto.

The following reagents and materials are suitable for use in the disclosed methods. Aminoethanethiol (AC15377-0250, Fisher), chloroform (319988-500 mL, Sigma), and ethidium bromide (E1510-10 mL, Sigma) were used as received. Quantum Dots dispersed in toluene (7.2 nm evidot 490, Evident Technologies, Troy, N.Y.) were used for conjugation with SYRB 101 dye (succinimidyl ester). DNA 100 bp ladder (15628-019), SYBR Gold (S11494), PICOGREEN (P7581) and SYBR 101 (S21500) dyes from Invitrogen (Carlsbad, Calif.) were used without purification. Hydrogen tetrochloroaurate (III) trihydrate (203386, Sigma) and sodium citrate dihydrate (W302600, Sigma) were used for gold nanoparticle synthesis. 1,4-Dithio-DL-threitol (DTT; D5545, Sigma) was used for the cleavage of oxidized thiolated oligonucleotides and bio-barcode release. DNA oligonucleotides and 6-Carboxyfluorescein (6-FAM) labeled oligonucleotides were purchased from Integrated DNA Technologies (Coralville, Iowa). Sulfosuccinimidyl 4-N-maleirnidomethyl cyclohexane-l-carboxylate (sulfo-SMCC; 22122, Pierce, Wis.) was used as a cross-linker between thiolated DNA and amine-coated MMPs.

### Example 1

### Bio-Barcode Assay

The MMP and AuNP set was constructed as described in H. D. Hill, R. A. Vega, and C. A. Mirkin, “Nonenzymatic detection of bacterial genomic DNA using the bio bar code assay,” Anal Chem, vol. 79, pp. 9218-23, Dec. 1, 2007, and D. Zhang, D. J. Carr, and in E. C. Alocilja, “Fluorescent bio-barcode DNA assay for the detection of Salmonella enterica serovar Enteritidis,” Biosens Bioelectron, vol. 24, pp. 1377-81, Jan. 1, 2009, the contents of which are incorporated by reference in their entirety.

Briefly, the target gene for hybridization was ethanol-precipitated and amplified with PCR. The target gene was identified and two probes were created, one probe, 5′-probe binds to the 3′ end of the target gene sequence, the 3′-probe binds to the 5′ end of the target gene sequence. The MMP was conjugated to the 5′-probe via SMCC conjugation. The 3′-probe and bio-barcode sequences were attached to the AuNP through ligand exchange of the capping agent for the thiolated DNA as in Hill, 2006. Both 3′ and 5′-probes bind to the complement of the gene target and use the target gene as a linker between the MMP and the AuNP. The BBC with the desired sequence and a 6-FAM (6-carboxyflorcein) and thiol modification may be ordered from any commercial sources, which is readily available to those skilled in the art. The 6-FAM is a fluorophore that is attached one end of the BBC, the other end of the BBC is modified with a thiol, a linker. The thiol group allows for the BBC attachment to the AuNP via ligand exchanged. The BBC for standard BCA detection without hybridization amplification was 5′ labeled with 6-FAM (λₑₓ=495 nm, λₑₘ=517 nm). The BCA works by having the complementary target gene sequence hybridize to both the MMP and the AuNP forming a link between the two particles. The MMPs allow the solution to be magnetically purified and concentrated. The concentrated solution will only contain AuNPs if they are connected to the MMPs via a hybridization linkage through the complementary target sequence. All DNA including the 6-FAM labeled BBC is released from the MMP-target-AuNP complexes with DTT (0.1 M) at 95° C. for 10 minutes. The DTT treated solution is centrifuged and the fluorescent supernatant containing the released 6-FAM labeled BBC is measured with the VICTOR plate reader system (Perkin Elmer; Waltham, Mass.). The BBC for co-polymerization hybridization according to the present disclosure was not 6-FAM labeled.

### Example 2

### Sequence Design

The twenty-two base BBC sequence published by Mirkin et al. (H. D. Hill, R. A. Vega, and C. A. Mirkin, “Nonenzymatic detection of bacterial genomic DNA using the bio bar code assay,” Anal Chem, vol. 79, pp. 9218-23, Dec. 1, 2007) was modified to contain two unique and distinct 11 base pair regions and designated as the CPH-probe. When a single copy of CPH-probe and a single copy of BBC hybridize, 50% of each oligonucleotide is hybridized to the other molecule creating ssDNA-dsDNA-ssDNA regions in the resulting molecular sequence. The unhybridized ssDNA regions allow for another molecule of ssDNA BBC to bind to the ssDNA overhang region of the hybridized CPH-probe, and vice versa for another ssDNA CPH Probe. The BBC and CPH-probe do not bind to themselves, nor do they bind to the genomic DNA (e.g., the assay target or genomic DNA of another organism). The CPH-probe sequence was generated by modifying the sequence of the BBC. The BBC sequence was modified to contain two 11 base regions: bases 1-11 (region 1) and bases 12-22 (region 2). The CPH-probe was designed by inverting the two regions, such that region 2 preceded region 1. The modified BBC target sequence was 5′-TTATT CGTAG CGTGA TGCCA AG-3′ (SEQ ID NO: 1) and the CPH-probe sequence was 5′-GCTAC GAATA ACTTG GCATC AC-3′ (SEQ ID NO: 2). The CPH-probe sequence resulted in an 11 bp overhang on both strands after hybridization. Ideal CPH-probe and BBC conditions are: 40-60% guanine (G) or cytosine (C) content, 18-36 bases in total length, equal lengths between the two, 50% bonding between two individual molecules, a 50% hybridization ratio (Tm) in the range 21-50° C., a “G” or “C” with 2 location from either end of both molecules, and no secondary structure formation occurring either individually or when mixed.

The ssDNA overhang portions on both the BBC and the CPH-probe allowed for further hybridization to other ssDNA portions of other molecules and subsequent long chain double stranded DNA (dsDNA) regions to form through co-polymerization. FIGS. 1D-1F generally illustrate the co-polymerization assembly scheme.

### Example 3

### Target Generation and Barcode Recovery

Genomic DNA was prepared as described previously in D. Zhang, D. J. Carr, and E. C. Alocilja, “Fluorescent bio-barcode DNA assay for the detection of Salmonella enterica serovar Enteritidis,” Biosens Bioelectron, vol. 24, pp. 1377-81, Jan. 1, 2009. The target sequence was PCR amplified and the product size was confirmed by gel electrophoresis and the PCR yield concentration was measured through spectrophometry (SmartSpec 3000, BioRad Laboratories). The PCR product was heated to 95° C. for 10 minutes and then serially diluted to 1 ng/μL. The target DNA (40 μL total volume) was added to 800 μg of MMPs and 40 μL of AuNPs in 200 μL, of assay buffer (10 mM PBS buffer, 0.15 M NaCl, 0.1% SDS, pH=7.4). Hybridization was carried out at 45° C. for 45 minutes. The solution was magnetically separated for 5 minutes and the supernatant removed. The solution was resuspended in 500 μL of assay buffer and washed five times removing unreacted AuNPs. Final resuspension was to a volume of 20 μL.

The final sample was heated to 95° C. for 10 minutes in 0.1 M DTT solution to dehybridize the target from the particles and to release the 6-FAM labeled bio-barcode from the AuNP. The solution was magnetically separated for 5 minutes and the supernatant transferred to a new micro-centrifuge tube. The supernatant containing BBC, target DNA and AuNPs was spun at 13,000×g for 30 minutes to pellet the AuNPs. The supernatant containing the BBC and target was removed and transferred into a new micro-centrifuge tube for readout.

### Example 4

### Quantum Dot Attachment

Five nanomoles of quantum dots (dₚ=7.2 nm, λₑₓ=470 nm, λₑₘ=490 nm) as received from Evident Technologies were dispersed in 5 mL of chloroform. Aminoethanethiol (AET) (0.5 M) in methanol was added drop-wise to the quantum dot solution until the AET began to flocculate. The head space was nitrogen flushed and vortexed. Ultrapure water was added to the suspension, and the sample was re-flushed with nitrogen and vortexed to promote phase exchange of the AET capped quantum dots as described in B. K. Pong, B. L. Trout, and J. Y. Lee, “Modified ligand-exchange for efficient solubilization of CdSe/ZnS quantum dots in water: a procedure guided by computational studies,” Langmuir, vol. 24, pp. 5270-6, May 20, 2008, the content of which is incorporated by reference in its entirety. The mixture was allowed to settle under gravity and the water soluble, AET functionalized QDs were suspended in the top aqueous layer. The aqueous layer was removed by micro-pipette and stored at 4° C. Phase exchange was confirmed by UV excitation (λ=254 nm) of reaction mixture. Absence of fluorescence in the chloroform fraction confirmed the absence of the QDs.

SYBR 101 dye (λₑₓ=480 nm, λₑₘ˜520 nm) was suspended in dimethyl sulfoxide (DMSO) at a concentration of 10 mg/mL. A volume of 10 μL (10 mg/mL) of the dye solution was added to 5 nmol of amine functionalized QDs in 1 mL buffer (0.1 M sodium phosphate, 0.15 M sodium chloride, pH 7.2). SYBR 101 contains an amine-reactive succinimidyl ester used for conjugation to primary amines. The amine-SYBR 101 reaction proceeded as outlined by the manufacture (Invitrogen S21500). Excess SYBR 101 was removed via centrifugation after QD attachment. The purified SYBR101-QD pellet was resuspended in 500 μL of phosphate buffer.

Fluorescent quantum efficiency of the AET capped quantum dots was lowered as a result of sulfur-metal surface binding. The QDs produced in these experiments exhibited a 68.5% decrease in quantum efficiency after water solubilization. Particles remained stable for more than three months at 4° C.

Dye fluorescence was maintained after conjugation to amine coated QDs. Both unconjugated and conjugated dyes were used to stain dsDNA molecules. Dye fluorescene is used only as a redundant checking mechanism while electrical detection methods are being optimized. Conjugated dye maintained 38% fluorescence after attachment seen in FIG. 2. The SYBR 101 only stained lanes (3,5, and 7) show a greater signal intensity than the respective SYBR 101-QD stained lanes (4,6, and 8).

The decrease in fluorescence is a result of the attachment of the QD to the SYBR 101 dye. The emission of the QDs is 480-500 nm and blocked by the ethidium bromide camera filter leaving the signal generated in lanes 4,6, and 8 a result of the SYBR 101 dye. The lowered signal from the SYBR 101 dye may be a result of the QDs acting as a quenching agent when in close proximity to DNA and SYBR 101 systems. of dsDNA (4,6,8) respectively. Gel: 2% w/v agarose, 30V, 2 hr in 1×TBE buffer.

### Example 5

### Target DNA Detection

The BBC was diluted in water and mixed with the CPH-probe in 100 μL of binding buffer (1×STE (sodium chloride-Tris-EDTA), 10× PICOGREEN). The solution was heated in a thermocycler to 95° C. for three minutes to promote dsDNA formation upon cooling to room temperature. The solution was centrifuged and transferred into a 96-well plate by micropipette. Fluorescent intensity was measured in 0.1 second readings using a fluorometer (Victor 1420, Perkin Elmer) (λₑₓ=485 nm, λₑₘ=520 nm).

### Example 6

### SPCE Biosensor Fabrication and Testing

All lanes run in the gel images were incubated with the indicated dye prior to be run in the gel. Gels were 2% w/v agarose to tris-boric-EDTA (TBE) buffer. Short wave UV illumination (λₑₓ=254 nm) was used to image all dyes in gel images. Images were taken with a CCD digital camera with a 540-660 nm ethidium bromide filter (Fotodyne CFW-1312M grayscale camera, 60-2030 filter).

Because other modifications and changes varied to fit particular operating requirements and environments will be apparent to those skilled in the art, the disclosure is not considered limited to the examples chosen for purposes of illustration, and covers all changes and modifications which do not constitute departures from the true spirit and scope of this disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. It is the intention, therefore, to be limited only as indicated by the scope of the claims appended hereto.

Accordingly, the foregoing description is given for clarity of understanding only, and no unnecessary limitations should be understood therefrom, as modifications within the scope of the disclosure may be apparent to those having ordinary skill in the art.

Throughout the specification, where the compositions, kits, processes, or apparatus are described as including components, steps, or materials, it is contemplated that the compositions, processes, or apparatus can also comprise, consist essentially of, or consist of, any combination of the recited components or materials, unless described otherwise. Component concentrations expressed as a percent are weight-percent (% w/w), unless otherwise noted. Numerical values and ranges can represent the value/range as stated or an approximate value/range (e.g., modified by the term “about”). Combinations of components are contemplated to include homogeneous and/or heterogeneous mixtures, as would be understood by a person of ordinary skill in the art in view of the foregoing disclosure.

1. A method for making a double-stranded DNA molecule, the method comprising:
(a) providing a population of a first single-stranded DNA (ssDNA) oligonucleotide having a first region and a second region in the 5′ to 3′ direction, wherein the first region is 5′ in position relative to the second region;
(b) providing a population of a second single-stranded DNA (ssDNA) oligonucleotide having a third region and a fourth region in the 3′ to 5′ direction for hybridization with the first ssDNA oligonucleotide, wherein:
(i) the third region is 3′ in position relative to the fourth region;
(ii) the second region is complementary to the third region, and the first region is complementary to the fourth region; and
(iii) the first region and the fourth region become overhangs when the second region and the third region hybridize together, and the second region and the third region become overhangs when the first region and the fourth region hybridize together; and
(c) hybridizing the population of the first ssDNA oligonucleotide and the population of the second ssDNA oligonucleotide under a predetermined hybridization condition to form a double-stranded DNA (dsDNA) molecule, the dsDNA molecule having repetitive first ssDNA oligonucleotide units on one strand and repetitive second ssDNA oligonucleotide units on the other strand.

2. The method of claim 1, wherein the first ssDNA oligonucleotide and the second ssDNA oligonucleotide each independently have between 8 to 60 bases.

3. The method of claim 1, wherein:
(i) the first ssDNA oligonucleotide comprises a sequence according to SEQ ID NO: 1; and
(ii) the second ssDNA oligonucleotide comprises a sequence according to SEQ ID NO: 2.

4. The method of claim 1, wherein the second region is complementary to the third region by at least 85%, and the first region is complementary to the fourth region by at least 85%.

5. The method of claim 1, wherein the dsDNA molecule has a average length of at least 40 base pairs.

6. The method of claim 1, wherein the first ssDNA oligonucleotide does not hybridize with itself and the second ssDNA oligonucleotide does not hybridize with itself.

7. The method of claim 1, wherein a ratio of the first ssDNA oligonucleotide to the second ssDNA oligonucleotide ranges between 0.1 and 10.

8. The method of claim 1, comprising gradually adding the population of the second ssDNA oligonucleotide to the population of the first ssDNA oligonucleotide.

9. The method of claim 1, comprising providing the population of the first ssDNA oligonucleotide as a plurality of subsamples to which a different amount of the second ssDNA oligonucleotide is added for each subsample.

10. The method of claim 1, further comprising:
(d) detecting the dsDNA molecule.

11. The method of claim 10, wherein part (d) comprises optically detecting the dsDNA molecule.

12. The method of claim 10, wherein part (d) comprises electrically detecting the dsDNA molecule.

13. The method of claim 1, wherein part (a) comprises:
(i) providing a bio-barcode nanoparticle probe comprising: (A) a nanoparticle core, (B) a first binding pair member immobilized on the nanoparticle core, the first binding pair member being capable of binding to a target analyte, and (C) the first ssDNA oligonucleotide immobilized on the nanoparticle core; and
(ii) releasing the first ssDNA oligonucleotide from the nanoparticle core to provide the population of the first ssDNA oligonucleotide.

14. The method of claim 13, wherein the target analyte is a target DNA sequence; and the first ssDNA oligonucleotide and the second ssDNA oligonucleotide do not hybridize with the target DNA sequence.

15. The method of claim 13, wherein the first ssDNA oligonucleotide is directly attached to the nanoparticle core in part (a)(i).

16. The method of claim 13, wherein the bio-barcode nanoparticle probe further comprises (D) a bio-barcode complement single-stranded DNA (BBC complement ssDNA) oligonucleotide directly attached to the nanoparticle core, the BBC complement ssDNA being capable of hybridization with the first ssDNA; and the first ssDNA oligonucleotide is immobilized on the nanoparticle core in part (a)(i) via conjugation with the BBC complement ssDNA.

17-20. (canceled)

21. The method of claim 1, wherein part (a) comprises:
(i) providing an analyte conjugate comprising:
(A) a bio-barcode nanoparticle probe comprising: a nanoparticle core, a first binding pair member immobilized on the nanoparticle core, the first binding pair member being capable of binding to a target analyte, and the first ssDNA oligonucleotide immobilized on the nanoparticle core,
(B) a magnetic microparticle probe comprising: a magnetic microparticle core, and a second binding pair member immobilized on the magnetic microparticle core, the second binding pair member being capable of binding to the target analyte, and
(C) the target analyte bound to both the first binding pair member of the bio-barcode nanoparticle probe and the second binding pair member of the magnetic microparticle probe; and
(ii) releasing the first ssDNA oligonucleotide from the nanoparticle core to provide the population of the first ssDNA oligonucleotide.

22. The method of claim 21, wherein providing the analyte conjugate comprises contacting the target analyte with the bio-barcode nanoparticle probe and the magnetic microparticle probe under conditions sufficient to bind the target analyte to the first binding pair member of the bio-barcode nanoparticle probe and the second binding pair member of the magnetic microparticle probe.

23. The method of claim 21, wherein the target analyte is a target DNA sequence; and the first ssDNA oligonucleotide and the second ssDNA oligonucleotide do not hybridize with the target DNA sequence.


SEQUENCE LISTING (3 sequences)

SEQ ID NO: 1 - DNA, 22 bp, Artificial Sequence
  Synthetic oligonucleotide
ttattcgtagcgtgatgccaag

SEQ ID NO: 2 - DNA, 22 bp, Artificial Sequence
  Synthetic oligonucleotide
gctacgaataacttggcatcac

SEQ ID NO: 3 - DNA, 22 bp, Artificial Sequence
  Synthetic oligonucleotide
cttggcatcacgctacgaataa